(19) United States
(12) Patent Application Publication
(10) Pub. No.: US 2019/0298739 A1
(43) Pub. Date: Oct. 3, 2019

(54) TREATMENT OF CALCIFIC AORTIC VALVE DISEASE

(71) Applicant: University of Manitoba, Winnipeg (CA)

(72) Inventor: Rizwan A. Manji, Winnipeg (CA)

(21) Appl. No.: 16/367,964

(22) Filed: Mar. 28, 2019

Related U.S. Application Data
(60) Provisional application No. 62/649,905, filed on Mar. 29, 2018.

(51) Int. Cl. A61K 31/573; C07K 16/28; A61P 9/00

(52) U.S. Cl. CPC A61K 31/573; A61K 45/06; A61P 9/00; C07K 16/2887

(57) ABSTRACT

A method of treating calcific aortic valve disease (CAVD), such as aortic valve sclerosis or stenosis, comprises administering an immunosuppressive drug or therapy to CAVD patient to slow or prevent progression of CAVD. Individuals who do not have cardiovascular disease may benefit particularly from the method described. The presence of antibody or Infiltration of immune cells into aortic valve tissue may be slowed or prevented using the method described. Uses and kits for addressing CAVD are also provided. Immunosuppressive drugs or therapies comprise but are not limited to steroids, glucocorticoids methotrexate, mycophenolate mofetil, Rituximab, anti-CD 20 monoclonal antibodies, anti-macrophage monoclonal antibodies, azathioprine, cyclosporine, leflunomide, cyclophosphamide, chlorambucil, nitrogen mustard, hydroxychloroquine, sulfasalazine, tacrolimus, or rapamycin.

### CROSS REFERENCE TO RELATED APPLICATIONS

This application claims the benefit of priority of U.S. Provisional Patent Application No. 62/649,905, filed Mar. 29, 2018, which is herein incorporated by reference.

### FIELD

The present disclosure relates generally to treatment, prophylaxis, or preventing progression of calcific aortic valve disease (CAVD).

### BACKGROUND

The aortic valve is a one-way valve between the left ventricle and the aorta. The aortic valve is covered by endothelium on both the aortic and ventricular sides and the valve itself has three layers: fibrosa, spongiosa and ventricularis, from the aortic side towards the ventricular side of the valve. Aortic stenosis is the third most common heart disease in Western world, behind coronary artery disease (CAD) and hypertension.

Calcific aortic valve disease (CAVD) is a broad category that encompasses a continuum of conditions, ranging from the mild aortic valve thickening seen in aortic sclerosis, to mild, moderate and severe aortic sclerosis, and to calcific aortic stenosis (CAVS), which at a severe level impairs valve motion. CAVD may be estimated to impact 100 million people in the developed world by 2050. A number of clinical risk factors are associated with hemodynamic progression of CAVD, including older age, male gender, hypercholesterolemia, higher body mass index, smoking, elevated serum LDL cholesterol, diabetes, elevated creatinine, renal failure, coronary artery disease, left ventricular mass index, calcium supplementation, elevated serum calcium, and mitral annular calcification (Freeman & Otto, 2005).

Calcific Aortic Valve Disease (CAVD) may be secondary to atherosclerosis. The analysis of Freeman & Otto (2005) compared histopathologic features and clinical risk factors found in CAVD versus atherosclerosis, and found that both conditions had certain features in common, while others were distinctly different. For example, the prominent cell type impacted in CAVD was found to be the fibroblast, while smooth muscle was found to be the prominent cell type in atherosclerosis. In terms of clinical risk factors hypertension, diabetes and endothelial dysfunction contributed more to the risk of atherosclerosis than of CAVD. The comparisons showed clear distinctions between the conditions, establishing that separate mechanisms are involved.

Degenerative calcific aortic valve stenosis (CAVS) is a common condition, with about 3% of the population over the age of 75 having the disease. The number of patients having hemodynamically significant CAVS is expected to increase over time from 1 million people in the United States at present to over 4.5 million in 2030 as the population continues to age. Currently there is no pharmacological treatment available and the only therapy is surgical aortic valve replacement (AVR), which may not be possible in certain patients, due to advanced age, frailty and significant co-morbidities. Individuals with kidney problems needing dialysis, heart rhythm problems needing a permanent pacemaker, or other health complications are not good candidates for surgery. The costs for such a surgery can be estimated at $100,000 within the first year of surgery. The disease progresses along a gradient from mild and moderate, to severe forms of CAVS.

Even if surgical aortic valve replacement (AVR) or transcatheter AVR are possible for treatment of more advanced cases of CAVS, there is still a high rate of stroke and major adverse cardiac events reported at the 3-year follow-up point, which impacts quality of life for patients and costs for the healthcare system. It would be beneficial if the pathogenesis of CAVS was better understood, and if pharmacological therapies could be devised to slow or prevent the progression of aortic valve sclerosis or milder forms of aortic stenosis toward mild, moderate, and severe aortic stenosis. Slowing progression of aortic sclerosis toward stenosis, and/or slowing the progression of mild and moderate stenosis toward the severe form that requires surgery would reduce the incidence of severe symptomatic aortic stenosis and reduce overall expenditures as a result of surgery. In the elderly population, which is usually the group experiencing CAVD and the progression to CAVS, a slowed progression, and avoidance of surgery, would have a significant and positive impact on quality of life in these later years.

Insights into the pathogenesis of CAVD, and CAVS in particular, have implicated lipid accumulation and an associated inflammatory response in aortic valve tissue. Histological examinations of human CAVS valves have identified various inflammatory cells including macrophages, B cells and T cells. While lipids, such as LDL cholesterol levels, are likely to be important factors, there are unknown factors in the pathogenesis of CAVS that lead some individuals with high lipid levels to have the fibrosis and calcification seen in the aortic valve. Multiple randomized control trials that have lowered lipid levels have shown negligible change in the rate of progression of CAVS. Additionally, many patients with CAVS do not have atherosclerotic disease elsewhere, and there are patients with atherosclerotic disease that do not have CAVS. The above observations suggest that other factors, besides just lipid accumulation, are important in the pathogenesis of CAVS.

There is no drug that slows the progression of aortic sclerosis or aortic stenosis per se, although treatments for atherosclerosis may be indicated in individuals with coincident atherosclerosis. The efficacy of atherosclerotic drugs in alleviating aortic valve sclerosis or stenosis is not established.

Immune cells are known to be present in stenotic cardiovascular tissues. Wallby et al. (2013) found macrophages infiltration and chronic inflammation in valves from patients with rheumatic and non-rheumatic aortic stenosis, with no differences attributable to the extent of deposition of apolipoprotein. Natorska et al. (2016) showed B cells to be present within aortic valves in patients with aortic stenosis. Hourai et al. (2017) found IgG4-positive cell infiltration in cardiovascular surgical samples, such as in the aortic valve from aortic stenosis and in the aortic wall from aortic aneurysm.

It is desirable to provide a method for treatment, prophylaxis, or for slowing or preventing the progression of calcific aortic valve disease (CAVD).

### SUMMARY

Described herein are methods for treatment, prophylaxis, or preventing the progression of calcific aortic valve disease (CAVD), which encompasses treatment of aortic sclerosis and calcific aortic stenosis (CAVS). Associated uses and kits are described. It has been found that human CAVD, such as CAVS, can be secondary to an immune response to antigen(s) expressed on the aortic valve. Treatment with immune suppression can deter progression from mild to moderate or severe forms of sclerosis and stenosis. By establishing that CAVD is an auto-immune process, similar to other autoimmune processes such as systematic lupus erythematosus (SLE), rheumatoid arthritis and others, the inventor has revealed a new form of treatment, thereby providing an alternative to disease progression and surgery.

It is an object of the present disclosure to provide a treatment for CAVD, such as aortic sclerosis or CAVS.

There is provided herein a method of treatment, prophylaxis, or preventing progression of calcific aortic valve disease (CAVD) in a patient with CAVD, by administering to the patient an immunosuppressive drug or therapy.

Further, there is provided a use of an immunosuppressive drug or therapy for the treatment, prophylaxis, or preventing progression of calcific aortic valve disease (CAVD), or for use in preparation of a medicament for such treatment, prophylaxis, or preventing progression, in a patient with CAVD.

Additionally, there is provided a kit for the treatment, prophylaxis, or preventing progression of calcific aortic valve disease (CAVD), wherein the kit comprises an immunosuppressive drug or therapy together with instructions for use in a patient with CAVD, according to the uses described herein.

There is also described herein a composition for use in a method for the treatment, prophylaxis, or prevention of progression of calcific aortic valve disease (CAVD) in a patient with CAVD, which composition comprises an immunosuppressive drug and a pharmaceutically acceptable excipient.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a PRIOR ART diagram of the structural features of a typical valve section.

FIG. 2 shows a valve section indicating the location of collagen (C) and elastin (E).

FIG. 3 shows two negative control sections according to Example 3, which are an aortic insufficiency valve (Panel A), and a pig valve (Panel B).

FIG. 4 shows an IgG staining of an aortic valve section having infiltrating cells.

FIG. 5 shows sections of aortic stenosis valves as per Example 3.

FIG. 6 shows two sections of aortic stenosis valves, as described in Example 3, illustrating IgG infiltration of arterioles, as well collagen (C) and Elastin (E).

FIG. 7 shows Western blot analysis of aortic stenosis valves, as described in Example 6, illustrating an autoimmune component to aortic stenosis.

### DETAILED DESCRIPTION

Generally, the present disclosure provides a method for treatment or prevention of progression of calcific aortic valve disease (CAVD), such as aortic sclerosis or calcific aortic valve stenosis (CAVS), by administration of an immunosuppressive drug or therapy to the patient in an amount effective to reduce or prevent autoimmune attack of the valve. Uses for such drugs or therapies in prevention or prophylaxis of the disease, as well as kits for carrying out the method are provided herein.

There are many autoimmune diseases where there may be multiple autoantigens involved and/or responsible, such as rheumatoid arthritis, systematic lupus erythematosus (SLE or lupus), and inflammatory bowel disease. However, without one or more specifically identified antigen it is accepted that an immune process leads to damage of the subject organ or tissue, which can be treated with immunosuppression.

In an autoimmune disease, a patient's own antibodies, such as IgG antibodies, bind to their own tissues. There is no new antigen expressed by the patient to initiate the autoimmune attack, but rather the patient forms antibodies to antigens that had been previously expressed in tissue, but which had not previously invoked antibody formation. Autoimmune diseases are often seen more frequently in females.

Similarly, the treatment of CAVS with immunosuppression may be used to alleviate the disease and slow or stop progression of the mild form to the severe form. The findings presented herein, showing antibody bound to the valve, indicate that use of immunosuppressive drugs or immune system suppression by other methods may be used.

There is provided herein a method of treatment, prophylaxis, or preventing progression of calcific aortic valve disease (CAVD) in a patient with CAVD, by administering to the patient an immunosuppressive drug or therapy. For example, the method may be used in certain patients where the CAVD is identified as mild to moderate aortic valve stenosis. An exemplary patient may be one that does not have a cardiovascular disease, and in particular does not have peripheral vascular disease, coronary artery disease, or a history of stroke. The method may be particularly useful in female patients.

The immunosuppressive drug or therapy may comprise, but need not be limited to a steroid, a glucocorticoid, a disease-modifying antirheumatic drug, a monoclonal antibody, or combinations of these. Specific non-limiting examples of drugs or therapies include steroids, glucocorticoids, methotrexate, mycophenolate mofetil, Rituximab, an anti-CD 20 monoclonal antibody, an anti-macrophage monoclonal antibody, azathioprine, cyclosporine, leflunomide, cyclophosphamide, chlorambucil, nitrogen mustard, hydroxychloroquine, sulfasalazine, tacrolimus, or rapamycin.

Uses of immunosuppressive drugs or therapies are described herein for the treatment, prophylaxis, or preventing progression of calcific aortic valve disease (CAVD) in a patient with CAVD, or for the preparation of a medicament for one or more of these purposes. Optionally, the CAVD to be treated in the patient may be mild to moderate aortic valve stenosis. Optionally, the patient may be one that does not have a cardiovascular disease such as peripheral vascular disease, coronary artery disease, or a history of stroke. For example, the patient may be female. Specific non-limiting examples of the immunosuppressive drug or therapy that may be used are those listed above with regard to the method described.

A kit is described herein for the treatment, prophylaxis, or preventing progression of calcific aortic valve disease (CAVD). The kit comprises an immunosuppressive drug or therapy together with instructions for use in a patient with CAVD, which patient may optionally be one that has mild to moderate aortic valve stenosis.

Compositions are described herein for use, or for use in a method, for the treatment, prophylaxis, or prevention of progression of calcific aortic valve disease (CAVD) in a patient with CAVD, and which may specifically be a patient with aortic valve sclerosis or stenosis (such as mild to moderate stenosis for example). The composition comprises an immunosuppressive drug and a pharmaceutically acceptable excipient.

The composition for use may comprise one or more immunosuppressive drug or therapy selected from the following non-limiting group: steroids, glucocorticoids methotrexate, mycophenolate mofetil, Rituximab, anti-CD 20 monoclonal antibodies, anti-macrophage monoclonal antibodies, azathioprine, cyclosporine, leflunomide, cyclophosphamide, chlorambucil, nitrogen mustard, hydroxychloroquine, sulfasalazine, tacrolimus, and rapamycin.

Treatment forAortic Stenosis. Therapeutic treatments can be considered to suppress the immune system of patients identified as able to benefit in treatment or prophylaxis of aortic stenosis. Such treatments include general immunosuppressive drugs currently used in other autoimmune diseases. Monoclonal antibodies or other biologics may be used.

Conventional small molecule drugs, biologic drugs such as monoclonal antibodies, intravenous immunoglobulin (IVIG), or plasmapheresis are treatments that may be used to achieve immune system suppression for mild to moderate aortic stenosis, as an alternative to surgery and to slow disease progression.

Treatment methods may include general approaches such as small molecule drugs to immune suppression, or more specific immunosuppression therapeutic approaches, such as monoclonal antibodies or other larger molecules often referred to as “biologics” like Rituximab, an anti-CD 20 monoclonal antibody, or other methods to remove antibody or prevent formation, such as other agents that may be known to prevent the expression of antigen.

Immunosuppressive drugs or therapy include steroids, glucocorticoids, mycophenolate mofetil (such as Cellcept®), cyclosporine, (such as Neoral®, Sandimmune®, or Gengraf®), tacrolimus, rapamycin, disease-modifying antirheumatic drugs (DMARDs) such as hydroxychloroquine, methotrexate (such as Rheumatrex®), sulfasalazine, azathioprine (such as Imuran®), and lefludomide (such as Arava®); and other cytotoxic drugs such as cyclophosphamide (such as Cytoxan®), chlorambucil (such as Leukeran®), or nitrogen mustard (such as Mustargen®); or an anti-CD 20 monoclonal antibody, such as Rituximab®, anti-macrophage monoclonal antibodies, anti t-cell monoclonal antibodies, or other monoclonal antibodies that address autoimmune attack.

By the term “immunosuppressive drug” any of these compounds or compositions containing them may be encompassed, including those not listed here, but know to have an immunosuppressive effect. Suitable drugs are ones that suppress, or reduce, the strength of the body's immune system. Drugs which may be used to make the body less likely to reject a transplanted organ, such as a liver, heart, or kidney (which may also be referred to as anti-rejection drugs) are encompassed here.

Optionally, candidate patients for whom the treatment may be effective can be identified by assessing appropriate clinical parameters indicative of autoimmune activity, such as antibody titre or other indices of inflammation.

As described herein, if one or more antigen(s) is expressed on aortic stenosis valves, then human antibody (and complement) may bind to the valve in immunohistochemistry studies. No examination of the specific question about whether human antibody binds to CAVS valves has previously been undertaken or found. The work described herein indicates that there is an antigen (auto-antigen) present on the valves leading to or as a result of the disease that could be addressed to treat, ameliorate, or lessen the harmful effects of the disease.

### EXAMPLES

### Example 1

Comparison with Observations in Bioprosthetic and Rheumatic Heart Valves.

Human tissue from CAVS valves, bioprosthetic heart valves (BHV) derived from pig or cow, removed from human patients, and rheumatic heart valves (RHV) from human patients were obtained at the University of Manitoba (Winnipeg, Canada) and examined for inflammatory cell infiltrate presence and distribution within the valves. By examining these bioprosthetic heart valves (BHV) removed from patients due to structural valve deterioration (SVD), and rheumatic heart valves (RHV) removed due to symptomatic heart disease, it has been shown that a similar gross pathology characterized by thickening/fibrosis and calcification is seen with bioprosthetic valves as is seen with Calcific Aortic Valve Disease (CAVS).

The BHV evaluated had antigens that elicited human antibody production resulting in an immune response against the valve. In addition, it is known that RHV have an antibody response against the valve due to molecular mimicry of valve proteins that are similar to bacterial proteins. Humans make antibodies against the streptococcus bacteria that causes rheumatic fever and these antibodies cross react with auto-antigens on the human's heart valve causing damage to the valve.

While there are similarities in gross pathology between BHV, RHV and CAVD, in particular CAVS, it is as yet unknown whether there are antigens (auto-antigens) that are present on CAVS to which humans have an immune response (humoral and/or cellular). Antigens may be expressed due to an exposure (such as an infection) as it is known that lipopolysaccharide from bacteria can stimulate valve interstitial cells, or the antigens may arise as part of an aging process similar to what happens to red blood cells as they age. Further, such antigens may arise from dietary sources, such as Neu5Gc being present on cancer cells or in atherosclerotic lesions. See Salama et al., 2017 or Samarj et al., 2014 for discussion of Neu5Gc.

Human antibody (IgG) was present in CAVS valves, as well as in BHV and RHV tissues, as revealed using immunohistochemistry.

These results indicate an autoimmune rejection may be at play in CAVS disease progression, in a manner that parallels the immune rejection process that may be observed in bioprosthetic valves, with mineralization of the valve. A pharmacological therapy resulting in immunosuppression for aortic valve sclerosis or stenosis, could treat or prevent the CAVD from worsening by preventing such autoimmune reactions. Such a treatment would represent a paradigm shift in the way early CAVD, and in particular CAVS, is currently managed by surgical valve replacement when the disease progresses.

### Example 2

Histological Evaluation of Valves from CAVS Patients.

In this Example, it is found that human calcific aortic stenosis (CAVS) is secondary to an immune response to one or more antigen expressed on the aortic valve. Treatment of patients to address the antigen specifically or to suppress the immune system can address CAVS. Immune suppression, especially in patients showing no significant signs of atherosclerotic disease, can be used as a therapeutic approach.

### BACKGROUND

Previous studies indicate that the ectopic mineralization of the aortic valve involves complex relationships with immunity. However, there is no clear evidence, to date, to show that clinical evolution of aortic stenosis involves an inflammatory process within the aortic valve. Innate and adaptive immunity may play a role in the development of CAVD. There is evidence that fibrocalcific remodeling of the aortic valve is associated with activation of the NF-kB pathway. The expression of TNF-α and IL-6 is increased in human mineralized aortic valves and promotes an osteogenic program as well as the mineralization of valve interstitial cells (VICs), the main cellular component of the aortic valve.

It is clinically accepted that lipids first infiltrate the valve tissue often in the vicinity of mineralized areas. In this regard, oxidized-LDL (ox-LDL) is a potent trigger of inflammation through Toll-receptors. Also, ox-LDL generates epitopes, which may activate adaptive immunity. In addition, the overexpression of lipoprotein-associated phospholipase A2 (Lp-PLA2), lipoprotein lipase (LPL), and 5-lipoxygenase (5-LO) in CAVD contributes to generate bioactive lipid-derived species, which amplify inflammation. These factors contribute to the activation of the NF-kB pathway. This leads to the observation of dense inflammatory infiltrates present in valves where oxidized lipids and ectopic mineralization are observed. Previous studies have noted that in about 15% of mineralized aortic valves osteogenic metaplasia is observed.

It has been documented that statins, although efficient to reduce clinical events in patients with atherosclerosis, are inefficient in treating CAVD. Previous randomized trials that have examined the role of statins in CAVD have concluded that a lipid-lowering strategy, through very efficient at reducing LDL plasma concentration to target values, failed at reducing the progression of aortic stenosis. These findings do not eliminate the potential role of lipid-derived factors in the pathobiology of CAVD but instead suggest that beyond lipid retention, other processes are at play and may promote, by a chain of events, the retention of lipids and their oxidation products, which may promote tissue remodeling and disease activity.

It has now been found, and is described herein, that CAVS can be related to an immune response caused by an antigen/antibody interaction on the valves which results in an inflammatory response. Examining the histology of CAVS valves shows evidence of inflammatory cell infiltrate though the extent of infiltrate was variable within a valve and between valves. Immunohistochemistry on the valves shows the presence of anti-human IgG bound to the valves. The extent of anti-lgG antibody binding was variable, but the pattern observed in the valves showed less binding in samples from male patients and more binding in samples from female patients.

Thus, CAVD, and in particular CAVS, can be treated as an auto-immune process by administering pharmacological therapy for aortic valve sclerosis or stenosis to prevent the disease from worsening, or slow the advancement of the disease.

Methods & Results.

By examining the histology of 5 CAVS valves, it was shown that inflammatory cells infiltrate though the valve. The extent of infiltration is variable within a valve and between valves. Immunohistochemistry on 2 of the valves shows the presence of IgG bound to the valves, which was unexpected.

In order to further examine histological samples, the presence of human antibody, such as IgG, can be evaluated in CAVS valves, and compared with BHV and RHV using immunohistochemistry. Comparing CAVS valves with BHV/RHV that are known to have an antigen/antibody interaction supports the assertion that CAVS is related to an autoimmune response to antigen present on the valves.

Histological examination is conducted on human CAVS, BHV and RHV valves for inflammatory cell infiltrate presence and distribution within the valves. Valves are formalin fixed in this, and in other Examples, unless otherwise stated.

Aortic stenosis valves are examined histologically and via immunohistochemistry. Results are compared to aortic insufficiency valves (negative control) or rheumatic heat disease valves (positive control). Rheumatic valves, are known to have an immune response to antigen on the valves and thus act as appropriate positive controls. As a result of examining aortic stenosis valves removed from individuals undergoing surgery, some variability of immune response is illustrated, as expected.

The demographic, co-morbidity (eg. other atherosclerotic disease), laboratory profile (eg. lipid profile), medication history, and echocardiographic information (gradients across valve and amount of insufficiency of valve) is evaluation for correlation to histology and immunohistochemistry findings. Variability of inflammatory infiltrate/immune response is to be expected in aortic stenosis valves. The clinical data of the patients from which the valves are derived correlates to the findings of the IgG staining.

There has previously been no indication that CAVD, such as aortic sclerosis or CAVS, could be treated as an autoimmune disease, by suppression of the immune response, such as in conditions like lupus, inflammatory bowel disease, or rheumatoid arthritis etc.

Discussion & Conclusion.

These data illustrate that CAVS is secondary to an immune response to antigens on the aortic valve. This suggests that CAVS is an auto-immune process (similar to other autoimmune processes such as lupus, rheumatoid arthritis and others) and thus one should consider pharmacological therapy (immunosuppression) for CAVD, and in particular for aortic valve sclerosis, or mild to moderate aortic stenosis, when it is appropriate and desirable, to prevent the disease from worsening. This therapy represents a paradigm shift in the way early CAVD, such as CAVS is currently managed. Immunosuppression in CAVD, and in particular in mild to moderate aortic stenosis, may prevent the further progression to severe aortic stenosis.

Importantly, the findings presented herein make a distinction between how a patient may be treated for atherosclerosis and how a patient may be treated when the disease condition is considered to be one attributable to autoimmunity. For the former condition, the treatment focus may be placed on lipid lowering drugs, while the latter treatment would focus on immune system suppression.

### Example 3

IgG Staining of Human Aortic Stenosis Valve Tissue.

This Example set out to the hypothesis that aortic stenosis is due in part to an autoimmune process as demonstrated by the binding of human IgG antibody to tissues on aortic stenosis valves.

Materials, Methods, and Results

Human stenotic valves were obtained from surgeries at hospitals affiliated with the University of Manitoba (Winnipeg Manitoba). Histological sections and staining were prepared using standard methods, and valve pathology was determined by a pathologist who read the slides. Aortic insufficiency valves were used as a negative control, and other appropriate controls were in place according to standard histopathological techniques. Valve sections were stained for human IgG. Staining was correlated to clinical factors known from confidentially managed patient data.

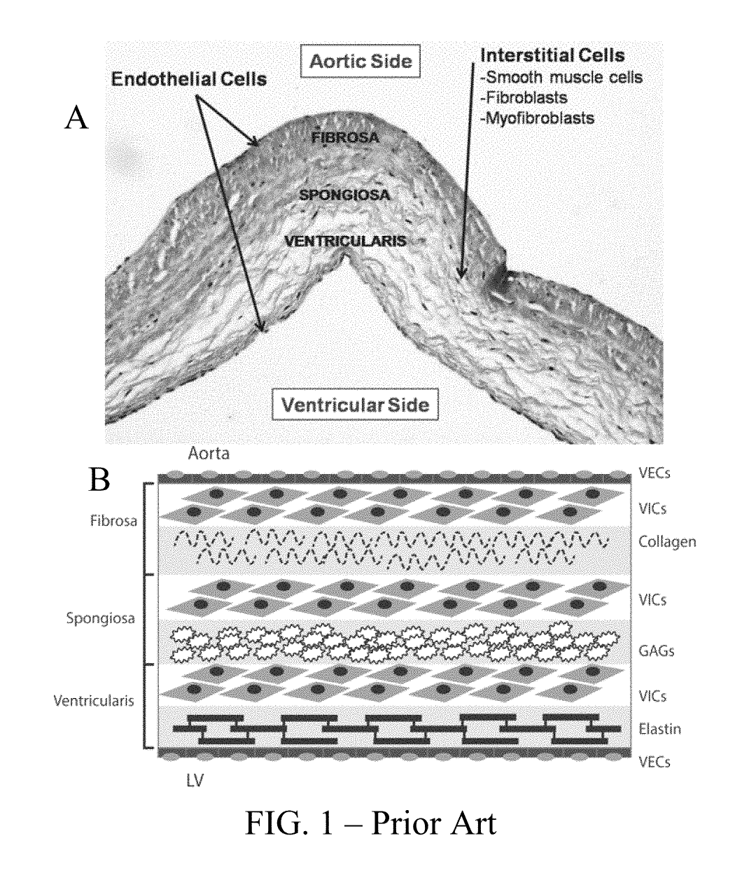

FIG. 1 illustrates a PRIOR ART diagram of the structural features of a typical valve section. Panel A shows a labelled section of valve according to the sections prepared in this Example, illustrating both the aortic side and left ventricular (LV) side of a section of valve tissue. Both aortic and ventricular sides had external endothelial cells, while smooth muscle cells, fibroblasts and myofibroblasts were contained within the tissue section. In Panel B, the fibrosa, spongiosa and ventricularis layers are schematically shown, together with the location of collagen and elastin layers, valve interstitial cells (VICs), valve endothelial cells (VECs) and glycosamino glycans (GAGs), adapted from Leopold (2012).

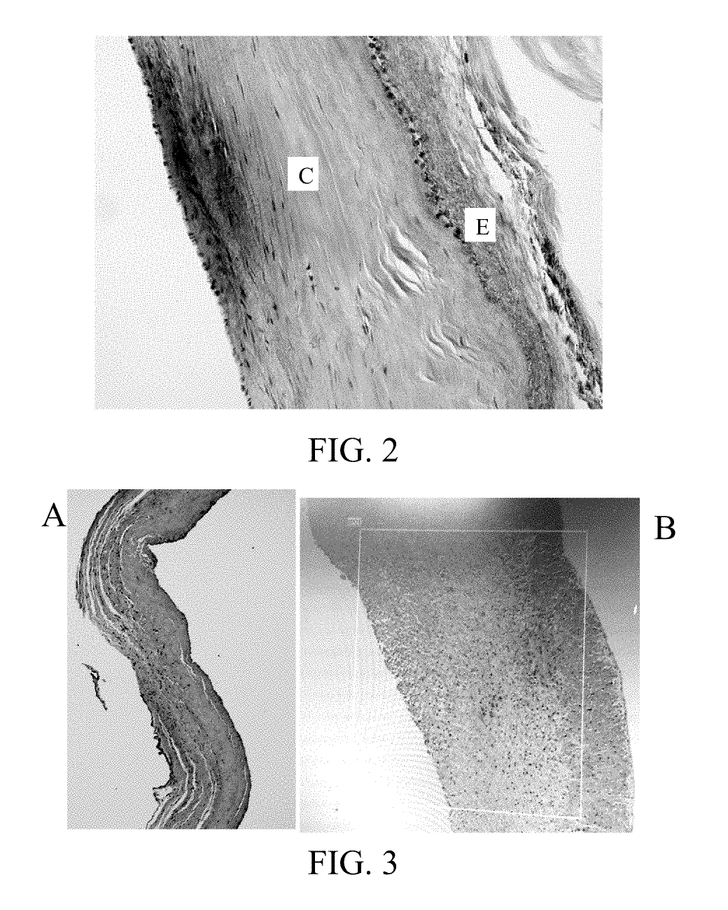

FIG. 2 shows a valve section indicating the location of collagen (C) and elastin (E).

FIG. 3 shows two negative control sections used in the evaluation: an aortic insufficiency valve (Panel A), and a pig valve (Panel B).

Sections were scored for the extent of staining found, with the score being compared to control sections. IgG staining of valve sections was compared to the same valve, but different sections, stained for anti-insulin antibodies, as a control to ensure that intensity scoring was not attributable to a sticky antibody effect.

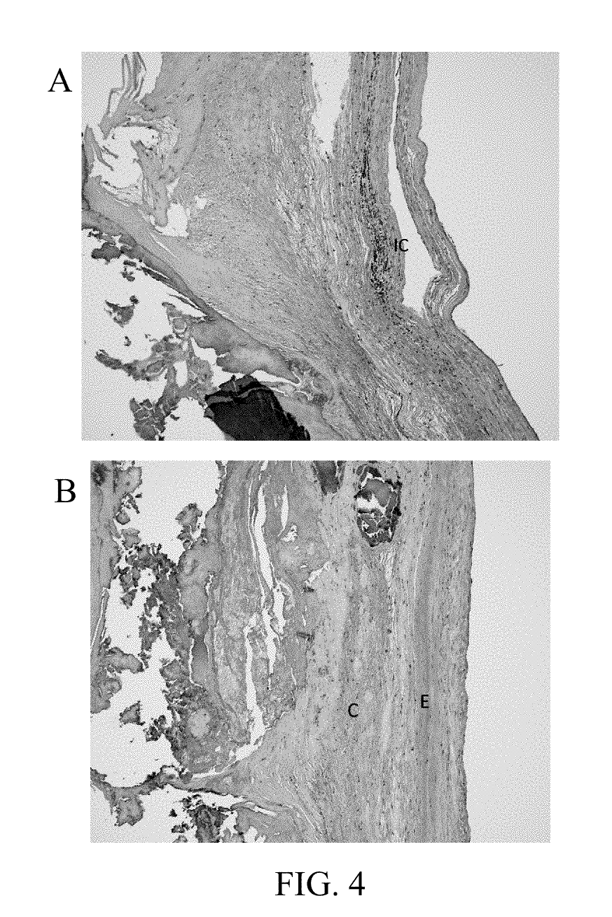

FIG. 4 shows an IgG staining of a section having infiltrating cells. Panel A shows a section where a clearly stained (initially a dark purple on colored slides) region marked as “IC” show a strong structural staining pattern for IgG, similar to staining observed in lupus. Panel B shows the staining of collagen (C) and elastin (E) layers in a valve section

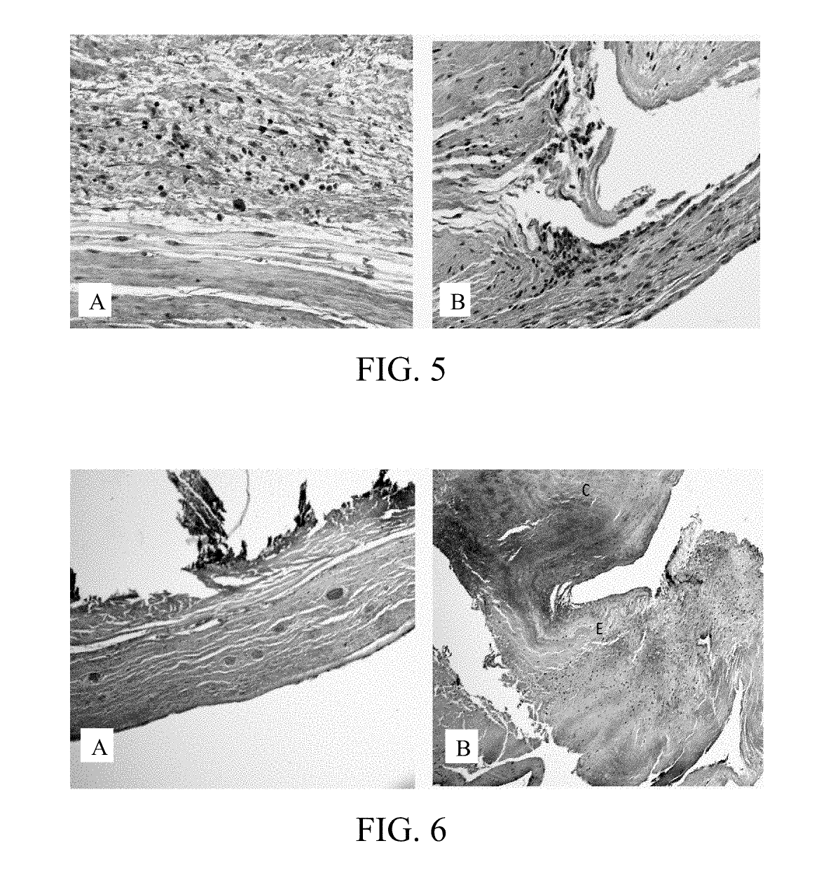

FIG. 5 shows sections of aortic stenosis valves. Panel A shows plasma cells and other inflammatory cells are stained darker. In Panel B, infiltrating lymphocytes can be seen with the darker stain.

FIG. 6 shows two sections of aortic stenosis valves. In Panel A, arterioles in the valve are stained. In Panel B, IgG on collagen (C) and Elastin (E) show clear IgG staining.

In the sections shown in the figures of this Example, the negative controls did not stain, and confirmed the absence of IgG, whereas a consistent staining of the valve tissue was seen in the region of the connective tissue. Staining of collagen and elastin, in a manner that is similar to and consistent with staining seen in lupus tissue sections show that IgG is present in the valves of patients with aortic stenosis.

Clinical parameters were correlated to staining scores of valve sections. For those sections exhibiting any staining with a score greater than 1, versus no staining (score of 0) for IgG. Of the 48 valves stained in total, 39 of these were scored (81%) had staining, while the remaining 9 valves showed no staining, and had a score of 0.

For the 9 valves with no staining, 7 of these 9 patients (78%) were male. Notably, autoimmune diseases are more common in females. The average age of the patients having valves with no staining was 71 years. The peak gradient across the valve for the no staining group was 50 mmHg, while the mean gradient across the valve for the no staining patients was 31 mmHg. All 9 of 9 (100%) patients without staining had cardiovascular (atherosclerotic) disease, and/or risk factors for same which include hypertension, diabetes, smoking, coronary artery disease, perivascular disease, cardiovascular disease, and hyperlipidemia.

For the 39 valves with visible staining, 15 of 39 (38%) are male, whereas most patients with staining are females (24 of 39). The average age of patients with staining was 74 years old. Peak gradient across the valve for the staining patients was 73 mmHg, and mean gradient across the valve for the staining patients was 43 mmHg. Of these patients, 33 of 39 (85%) have cardiovascular (atherosclerotic) disease or one of the risk factors therefor. Of the patients assessed, 50% had established atherosclerotic disease defined as: (i) coronary artery disease based on angiogram data or having a coronary artery bypass graft (CABG), (ii) peripheral vascular disease based on medical history; or (iii) cerebrovascular disease based on history of stroke or transient ischemic attack which is also known as a ministroke.

These results indicate that aortic stenosis results in autoimmune attack for a certain sub-section of patients, which tend to be female, and which need not have had any cardiovascular risk factors. These data support that aortic stenosis may be an autoimmune disease that would respond to immune suppression in certain patients.

There is no known reason to have human IgG antibody bind to human aortic valve tissue. IgG antibody staining to specific structures, in particular collagen and elastin, suggesting that the antigen to which IgG is binding is present on these tissues, which bears similarity to the situation seen in other autoimmune diseases, such as lupus.

Clinical parameters show that the staining occurs mostly in women which also supports the observation that aortic stenosis is an autoimmune process, given that autoimmune diseases are more common in women than men. The data indicate that treatment using method to slow the rate of progression of aortic valve sclerosis or mild stenosis using immune suppression may slow or stop the progress of the disease, as it progresses toward severe symptomatic stenosis with all of its attendant complications.

Treatment methods could include general approaches to immune suppression, such as steroids, methotrexate, mycophenolate mofetil, etc., or more specific immunosuppression approaches, (such as Rituximab, an anti-CD 20 monoclonal antibody) or other methods to remove antibody, such as other agents that may be known to prevent the expression of antigen.

### Example 4

Categorization of Immunostained Aortic Stenosis Valves

In this Example, there are 26 aortic stenosis valve samples that revealed strong staining, representative of immune cell infiltration. Out of the 26 patients showing the staining, only 13 (ie: 50%) had established atherosclerotic disease as defined according to parameters stated in Example 3. This compares to 9 patients having no IgG staining on their valves where 8 of the 9 patients (or 89%) had established atherosclerotic disease. There were 13 patients that had mild IgG staining on their valves and 8 of these (61%) had established atherosclerotic disease. Thus there is some separation based on established atherosclerotic disease.

Gender Differences.

The data revealed that males tended to have lower amounts of immune IgG antibody binding, as represented by staining, and more established atherosclerotic disease. Thus, the aortic stenosis pathophysiology may have been more influenced by serum lipids, and factors correlating with atherosclerosis in male patients. Female patients showing no atherosclerotic parameters nevertheless showed strong IgG antibody binding as evidenced by staining. Thus, female patients with no atherosclerotic disease may particularly benefit from immune suppression treatment to prevent the progression from mild CAVS to an intermediate or severe form of the disease. This is the first finding that cause and treatment of CAVS may differ based on gender.

Evaluating CAVS Patients Having Previous or Existing Autoimmune Conditions.

Autoimmune diseases tend to run in clusters. That is to say that if a patient has an autoimmune disease, that patient is more likely to also have another autoimmune disease.

The evaluation of the medical history of the patients, to see if there was documentation of confirmed autoimmune disease such as lupus, rheumatoid arthritis, inflammatory bowel disease, myositis, celiac disease, IgA disease and similar conditions was undertaken. These autoimmune conditions, as well as immune type disease such as asthma, eczema, hypothyroidism (as some hypothyroidism is due to autoimmune disease) were evaluated.

“Confirmed” and “Possible” categories for immune type diseases were addressed, and the results confirmed that treating CAVS as autoimmune disease in patients previously having a confirmed autoimmune disease was appropriate in determining treatment group. Of the 26 strongly stained valves, 5 of 26 (19%) had a “Confirmed” autoimmune disease which compares to 1/9 (11%) for patients with no staining of their valves, and 2/13 (15%) with mild staining of their valves.

For the combined “Confirmed” and “Possible” categories, of the 26 strong staining valves, 13/26 (50%) had Confirmed or Possible status for having had a previous autoimmune condition, which compares to 2/9 (22%) with no staining and 2/13 (15%) with mild staining. The stronger the staining, the more likely the patient was to have previously had an autoimmune condition.

### Example 5

Aortic Insufficiency Valves.

Aortic insufficiency valves were evaluated using methodologies described in the previous two examples. Twenty-five aortic insufficiency valves were stained to locate immune cell infiltration. In general, in the whole group of aortic insufficiency valves, there was less antibody (IgG) staining compared to the aortic stenosis group. However, there are aortic insufficiency valves that did show significant staining with antibody. A few of these valves were ones known to have had infection, to which the presence of antibody may be attributed. Further, there are valves from individuals that have a history of rheumatic heart disease. However, some aortic insufficiency valves exhibited staining, but without being attributable to these reasons. A double-check of the methodology affirmed that this result was an accurate observation. These data when taken together with the CAVS observations suggest the very interesting possibility that abnormal valves (whether the abnormality is attributable to aortic stenosis or to aortic insufficiency) have an immune component contributing to the deterioration or abnormality. All such valves eventually required surgical removal, thereby permitting inclusion in this Example.

Review of data by an independent pathologist confirmed that the results are not attributable to a methodological artifact.

Aortic insufficiency patients can also be treated with an autoimmune treatment regimen to treat or prevent aortic valve deterioration.

Therapeutic treatments can be considered to suppress the immune system of patients identified as able to benefit in treatment or prophylaxis of aortic insufficiency. Such treatments include general immunosuppressive drugs currently used in other autoimmune diseases.

### Example 6

Western Blot Analysis of Human Aortic Stenosis Valve Tissue.

Western blots were conducted and assessed on human aortic stenosis valve tissue to look for the presence of human IgG antibody in the aortic stenosis valves.

Ten human aortic stenosis valves were collected following surgeries conducted in Winnipeg, Canada. These valves were put into EDTA and subsequently frozen. Tissue from these valve samples were used for Western blot studies. A commercially purchased preparation of human IgG was used as the positive control. An actin control was used with the experiment to confirm that the same amount of total protein was added to each well, as determined by the intensity of the actin protein bands.

The tissues were thawed, and a Western blot assay was performed following standard protocols, by loading 50 ug of protein per well for the tissue samples and 0.5 ug of protein per well for the IgG positive control.

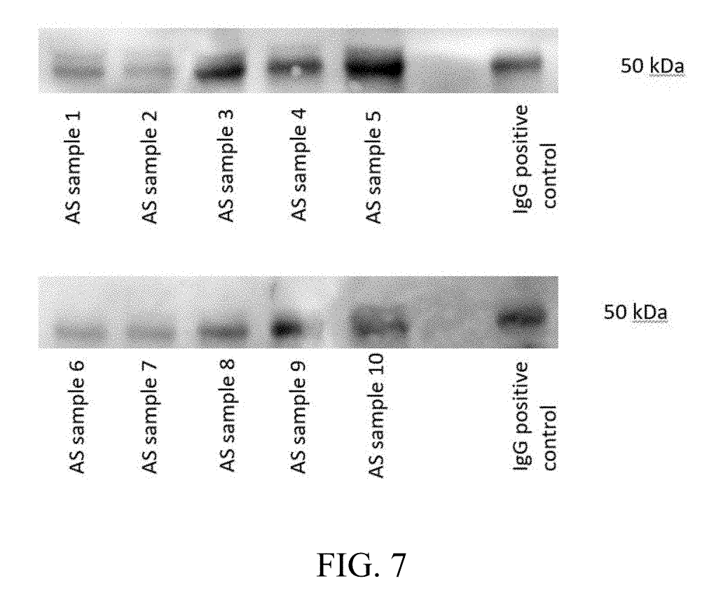

FIG. 7 indicates that the Western blot revealed the presence of human IgG antibody in the aortic stenosis valves, although there is variability in the amount of antibody present. Western blots (for human IgG) of 10 different aortic stenosis valves (AS Sample 1 to AS Sample 10) show the presence of Human IgG in the valves in variable amounts.

The Western blot data confirmed that there is human IgG antibody bound to the aortic stenosis valves. As these valves are the patient's own valves, any human IgG antibody bound to the valve would be abnormal and thus indicates there is an antigen being recognized on the valve. The antigen may be an auto-antigen. These date indicate that aortic stenosis has an autoimmune disease component to it, as illustrated the unexpected binding of human IgG antibody to the removed aortic stenosis valves.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

The following references are hereby incorporated by reference.
Capoulade et al. Autoantibodies and immune complexes to oxidation-specific epitopes and progression of aortic stenosis: Results from the ASTRONOMER trial. Atherosclerosis. 2017 May; 260:1-7. doi: 10.1016/j.atherosclerosis. 2017.03.013. Epub 2017 Mar. 9.
Chan et al. Effect of Lipid lowering with rosuvastatin on progression of aortic stenosis: results of the aortic stenosis progression observation: measuring effects of rosuvastatin (ASTRONOMER) trial. Circulation. 2010; 121:306-314.
Cote et al., Inflammation is associated with remodeling of calcific aortic valve disease. Inflammation. 2013. 36 (3):573-581.
Cowell et al. A randomized trial of intensive lipid-lowering therapy in calcific aortic stenosis. N Engl J Med. 2005; 352:2389-2397.
Edep et al., Matrix metalloproteinase expression in nonrheumatic aortic stenosis. Cardiovasc Pathol. 2000. 9(5):28-286.
Freeman & Otto. Spectrum of calcific aortic valve disease: pathogenesis, disease progression, and treatment strategies. Circulation. 2005 Jun. 21; 111(24):3316-26.
Gohlke-Bärwolf et al. Natural history of mild and of moderate aortic stenosis-new insights from a large prospective European study. Curr Probl Cardiol. 2013 September; 38(9):365-409. doi: 10.1016/j.cpcardiol.2013.06.003.
Grabell et al. Detection of Type VII Collagen Autoantibodies Before the Onset of Bullous Systemic Lupus Erythematosus. JAMA Dermatol. 2015; 151(5):539-543. doi:10.1001/jamadermatol.2014.4409
Hamatani et al. (2016). Pathological Investigation of Congenital Bicuspid Aortic Valve Stenosis, Compared with Atherosclerotic Tricuspid Aortic Valve Stenosis and Congenital Bicuspid Aortic Valve Regurgitation. PLOS ONE. 11. e0160208. 10.1371/journal.pone.0160208.
Hourai et al. IgG4-positive cell infiltration in various cardiovascular disorders—results from histopathological analysis of surgical samples. BMC Cardiovasc Disord. 2017 Feb. 3; 17(1):52. doi: 10.1186/s12872-017-0488-3.
Leopold. Cellular Mechanisms of Aortic Valve Calcification. Circulation: Cardiovascular Interventions. 2012; 5:605-614.
Lerman et al. Calcific Aortic Valve Disease: Molecular Mechanisms and Therapeutic Approaches. Eur Cardiol. 2015; 10(2): 108-112.
Mathieu et al., Innate and Adaptive Immunity in Calcific Aortic Valve Disease. Journal of Immunology Research. 2014. 2015:1-11. Miller et al. Calcific Aortic Valve Stenosis: Methods, Models, and Mechanisms. Circ Res. 2011 May 27; 108(11): 1392-1412.
Moreno et al. Increased macrophage infiltration and neovascularization in congenital bicuspid aortic valve stenosis, J. Thorac. Cardiovasc. Surg. 142 (2011) 895-901.
Moura et al. Rosuvastatin affecting aortic valve endothelium to slow the progression of aortic stenosis. J Am Coil Cardiol. 2007; 49:554-561.
Natorska et al., Presence of B cells within aortic valves in patients with aortic stenosis: Relation of severity of the disease Journal of Cardiology. 2016. 7:80-85.
Olsson et al., Accumulation of T lymphocytes and expression of interleukin-2 receptors in nonrheumatic stenotic aortic valves. J Am Coil Cardiol. 1994. 23(5):1162-70.
Otto et al., Characterization of the early lesion of degenerative valvular aortic stenosis. Histological and immunohistochemical studies. Circulation. 1994. 90:844-853.
Pibarot and Dumesnil. Prosthetic heart valves: selection of the optimal prosthesis and long-term management. Circulation. 2009 Feb. 24; 119(7):1034-48. doi: 10.1161/CIRCULATIONAHA.108.778886.
Reardon et al. Surgical or Transcatheter Aortic-Valve Replacement in Intermediate-Risk Patients. N Engl J Med. 2017 Apr. 6; 376(14):1321-1331. doi: 10.1056/NEJMoa1700456. Epub 2017 Mar. 17.
Reynolds et al. Cost-Effectiveness of Transcatheter Aortic Valve Replacement With a Self-Expanding Prosthesis Versus Surgical Aortic Valve Replacement. J Am Coil Cardiol. 2016 Jan. 5; 67(1): 29-38.
Rossebo et al. Intensive lipid lowering with simvastatin and ezetimibe in aortic stenosis. N Engl J Med. 2008; 359:1343-1356.
Roux-Lombard et al. Auto-antibodies as emergent prognostic markers and possible mediators of ischemic cardiovascular diseases. Clin Rev Allergy Immunol. 2013. 44(1):84-97.
Salama et al. Neu5Gc and α1-3 GAL Xenoantigen Knockout Does Not Affect Glycemia Homeostasis and Insulin Secretion in Pigs. Diabetes. 2017 April; 66(4):987-993.
Saleeb et al. Accelerated Degeneration of a Bovine Pericardial Bioprosthetic Aortic Valve in Children and Young Adults. Circulation. 2014; 130:51-60.
Samraj et al. A red meat-derived glycan promotes inflammation and cancer progression. Proc Natl Acad Sci USA. 2015 Jan. 13; 112(2): 542-547.
Steiner et al. Calcific aortic valve stenosis: Immunohistochemical analysis of inflammatory infiltrate. Pathology—Research and Practice 2012; 208(4):231-234. [00141] van Gils et al. Prognostic Implications of Moderate Aortic Stenosis in Patients With Left Ventricular Systolic Dysfunction J Am Coil Cardiol. 2017; 69:2383-92.
Wallby et al., T lymphocyte infiltration in non-rheumatic aortic stenosis: a comparative descriptive study between tricuspid and bicuspid aortic valves. Heart. 2002. 88(4):348-351.
Wallby et al., Inflammatory Characteristics of Stenotic Aortic Valves: A Comparison between Rheumatic and Nonrheumatic Aortic Stenosis. Cardiology Research and Practice. 2013. 2013:1-7.
Yutzey et al. Calcific Aortic Valve Disease: A Consensus Summary From the Alliance of Investigators on Calcific Aortic Valve Disease. Arterioscler Thromb Vasc Biol. 2014; 34:2387-2393.

1. A method of treatment, prophylaxis, or preventing progression of calcific aortic valve disease (CAVD) in a patient with CAVD, by administering to the patient an immunosuppressive drug or therapy.

2. The method of claim 1, wherein the CAVD is mild to moderate aortic valve stenosis.

3. The method of claim 1, wherein the patient does not have a cardiovascular disease selected from the group consisting of peripheral vascular disease, coronary artery disease, or a history of stroke.

4. The method of claim 1, wherein the patient is female.

5. The method of claim 1, wherein the immunosuppressive drug or therapy comprises a steroid, a glucocorticoid, a disease-modifying antirheumatic drug, or a monoclonal antibody.

6. The method of claim 1, wherein the immunosuppressive drug or therapy comprises a steroid, a glucocorticoid, methotrexate, mycophenolate mofetil, Rituximab, an anti-CD 20 monoclonal antibody, an anti-macrophage monoclonal antibody, azathioprine, cyclosporine, leflunomide, cyclophosphamide, chlorambucil, nitrogen mustard, hydroxychloroquine, sulfasalazine, tacrolimus, or rapamycin.

7. The method of claim 1, wherein the method is for preventing progression of calcific aortic valve disease (CAVD) in the patient.

8. The method of claim 1, wherein the method is for treating calcific aortic valve disease (CAVD) in the patient.

9. The method claim 8, wherein the CAVD is moderate aortic valve stenosis.

10. The method of claim 9, wherein the patient does not have coronary artery disease.

11. The method of claim 10, wherein the patient is female.

12. The method of claim 11, wherein the immunosuppressive drug or therapy comprises a steroid or a glucocorticoid.

13. The method of claim 11, wherein the immunosuppressive drug or therapy comprises a steroid, a glucocorticoid, methotrexate, mycophenolate mofetil, Rituximab, an anti-CD 20 monoclonal antibody, an anti-macrophage monoclonal antibody, azathioprine, cyclosporine, leflunomide, cyclophosphamide, chlorambucil, nitrogen mustard, hydroxychloroquine, sulfasalazine, tacrolimus, or rapamycin.

14. A kit for the treatment, prophylaxis, or preventing progression of calcific aortic valve disease (CAVD) comprising an immunosuppressive drug or therapy together with instructions for use in a patient with CAVD, according to the method of claim 1.

15. The kit of claim 14, wherein the CAVD is mild to moderate aortic valve stenosis.

16. The kit of claim 14, wherein the instructions are for use in a female patient.

17. A composition for use in a method for the treatment, prophylaxis, or prevention of progression of calcific aortic valve disease (CAVD) in a patient with CAVD, said composition comprising an immunosuppressive drug or therapy, and a pharmaceutically acceptable excipient.

18. The composition for use according to claim 17, wherein the CAVD is mild to moderate aortic valve stenosis.

19. The composition for use according to claim 17, wherein the immunosuppressive drug or therapy comprises a steroid, a glucocorticoid, methotrexate, mycophenolate mofetil, Rituximab, an anti-CD 20 monoclonal antibody, an anti-macrophage monoclonal antibody, azathioprine, cyclosporine, leflunomide, cyclophosphamide, chlorambucil, nitrogen mustard, hydroxychloroquine, sulfasalazine, tacrolimus, or rapamycin.

20. The composition for use according to claim 17, for use in a female patient.
